(19) United States
(12) Patent Application Publication
(10) Pub. No.: US 2012/0288925 A1
(43) Pub. Date: Nov. 15, 2012

(54) OPTICAL TRAPPING PARTICLES, ANGULAR OPTICAL TRAP SYSTEMS, METHODS OF MAKING, AND METHODS OF USE

(75) Inventors: Michelle D. Wang, Ithaca, NY (US); Christopher Deufel, Ithaca, NY (US)

(73) Assignee: Cornell University-Cornell Center for Technology Enterprise & Commercialization (CCTEC), Ithaca, NY (US)

(21) Appl. No.: 13/548,652

(22) Filed: Jul. 13, 2012

Related U.S. Application Data
(62) Division of application No. 11/970,121, filed on Jan. 7, 2008, now abandoned.
(60) Provisional application No. 60/883,666, filed on Jan. 5, 2007.

(51) Int. Cl. G01J 4/04; C12M 1/40; G01N 21/75; G01L 1/24; G01B 11/26

(52) U.S. Cl. 435/287.2; 356/365; 356/73; 422/69

(57) ABSTRACT

The present invention relates to an optical trapping particle including a birefringent crystalline particle having a body and a length extending between a first end and a second end, said particle comprising an optic axis perpendicular to the length of the body, wherein the length of the body is greater than the largest width dimension of the first or second ends. The present invention also relates to an optical trapping particle including an optically isotropic particle having a body and a length extending between a first end and a second end, said particle having an asymmetric cross-section, wherein the length of the body is from about 10 nanometers to about 10 micrometers and is greater than the largest width dimension of the first or second ends. Angular optical trap systems including the optical trapping particles, methods of making, and methods of use are also disclosed.

The present invention claims priority from U.S. Provisional Application Ser. No. 60/883,666, filed Jan. 5, 2007, which is hereby incorporated by reference in its entirety.

The subject matter of this application was made with support from the United States Government under NSF Grant No. DMR-0517349 and NIH Grant No. R01 GM059849. The U.S. Government has certain rights.

### FIELD OF THE INVENTION

The present invention relates to optical trapping particles, angular optical trap systems including the optical trapping particles, and methods of making and using the optical trapping particles.

### BACKGROUND OF THE INVENTION

Torque and rotation are critically important in biology. In particular, the bending and torsional properties of DNA influence numerous cellular processes, notably DNA compaction, replication, transcription, and protein-DNA binding. DNA elasticity regulates how proteins bend and twist DNA upon binding and how translocating molecular motors exert torque and force on their DNA substrates. In particular, molecular motors such as RNA polymerase can exert torque on their DNA substrate as they translocate and thereby twist DNA into a supercoiled state. Single molecule techniques have proven to be powerful approaches for the investigation of the response of DNA to mechanical stress; individual DNA molecules can be stretched and twisted under physiologically relevant conditions. To date the stretching and bending elasticities of DNA have been well characterized through measurements of the force-extension relation of DNA (Wang et al., Biophys. J., 72:1335-1346 (1997); Smith et al., Science, 258:1122-1126 (1992)). However, somewhat less is known regarding the torsional elasticity of DNA, at least in part due to difficulties in direct torque measurements.

The most prevalent method to twist DNA is to use magnetic tweezers to rotate a magnetic bead via rotation of a magnetic field (Strick et al., Science, 271: 1835-1837 (1996); Crut et al. Proc. Nat. Acad. Sci., 104:11957-11962 (2007)). Magnetic tweezers have been widely used to investigate DNA supercoiling and the action of various enzymes on supercoiled DNA, but without a direct measurement of torque (Charvin et al., V. Annu. Rev. Biophys. Biomol. Struc., 34:201-219 (2005)). Twisting of DNA can also be achieved by rotation of a micropipette cantilever (Leger et al., Phys. Rev. Lett., 83:1066-1069 (1999)). This approach, however, has not included torque detection. Viscous drag force and/or the angular Brownian motion of a bead have provided measurements of DNA torsional elasticity as well as torque during DNA structural transitions (Bryant et al., Nature, 424:338-41 (2003); Oroszi et al., P. Phys. Rev. Lett., 97:058301 (4 pages) (2006)). This approach requires taut DNA to minimize writhe and thus is more suited for measurements under high force (>15 pN).

Several other techniques have been demonstrated for rotating microscopic particles. These include the use of azimuthally asymmetric beams or combinations of beams to rotate non-spherical particles (O'Neil et al., Optics Letters, 27:743-745 (2002); Paterson et al., Science, 292:912-914 (1997); Bingelyte et al., Applied Physics Letters, 82:829-831 (2003)), the use of linearly or circularly polarized light to orient or apply torque to birefringent calcite particles (Friese et al., Nature, 394:348-350 (1998); La Porta et al., Phys. Rev. Lett., 92:190801 (4 pages) (2004)), or the use of magnetic fields to apply torque to free or optically trapped magnetic particles (Sacconi et al., Optics Letters, 26:1359-1361 (2001); Strick et al., Nature, 404:901-904 (2000)).

In most biophysical single molecule studies employing optical tweezers, a micron-sized particle chemically attached to a molecule of interest (e.g., DNA) serves as a handle to facilitate manipulation, calibration, and measurement in an optical trap. There have been a myriad of uses for optical traps in the field of single molecule biophysics, and the discussion below focuses on systems involving DNA. Briefly, an optical trap can be described as an instrument that focuses a collimated light, normally provided by a single mode laser, into a tight focus by a high numerical aperture (NA) objective lens to trap a dielectric particle. The principal forces involved in an optical trap are the scattering force (a result of reflection of the incident beam) and the gradient force, which is the force that actually does the trapping. The scattering force “is proportional to the light intensity and acts in the direction of the propagation of light” while the gradient force is “proportional to the spatial gradient in light intensity and acts in the direction of that gradient” (Svoboda, “Biological Applications of Optical Forces,” Annual Reviews, <www.annualreviews.org>, 249 (1994)). The diameter of the particles is on the order of the wavelength of light that is being used or somewhat smaller.

Conventional trapping particles are optically isotropic microspheres, which are only adequate for applying force. More specialized handles are needed to generate torque, and require either shape or optical anisotropy. Angular optical trapping instruments capable of direct application and detection of torque on optically anisotropic, birefringent microparticles or optically isotropic microparticles have been developed (Friese et al., Nature, 394:348-350 (1998); Bishop et al., Phys. Rev. A, 68:033802 (8 pages) (2003); La Porta et al., Phys. Rev. Lett., 92:190801 (4 pages) (2004); Bishop et al., Phys. Rev. Lett., 92:198104 (2004)). In these studies, the trapping particles were either fragmented materials with varying sizes and shapes (Friese et al., Nature, 394:348-350 (1998); Bishop et al., Phys. Rev. A, 68:033802 (8 pages) (2003); La Porta et al., Phys. Rev. Lett., 92:190801 (4 pages) (2004)) or microspheres with varying sizes and degrees of optical anisotropy (Bishop et al., Phys. Rev. Lett., 92:198104 (2004)). Torque is measured by detecting a change in angular momentum of the transmitted trapping beam. However, large heterogeneities in shape, size, and optical properties of such fragments complicate precise measurements on biological molecules. In addition, none of these studies demonstrated coupling of these particles to biological molecules. More regularly shaped particles, such as vaterite (Bishop et al., Phys. Rev. Lett., 92:198104 (4 pages) (2004)) or lysozyme crystals (Singer et al., Phys. Rev. E, 73:021911 (5 pages) (2006)), and compressed polystyrene beads (Oroszi et al., Phys. Rev. Lett., 97:058301 (4 pages) (2006)), have also been used to generate torque. However, biochemical coupling of these particles to biological structures either has yet to be shown (Bishop et al., Phys. Rev. Lett., 92:198104 (4 pages) (2004); Singer et al., Phys. Rev. E, 73:021911 (5 pages) (2006)) or was non-specific (Oroszi et al., Phys. Rev. Lett., 97:058301 (4 pages) (2006)).

The present invention is directed to overcoming these and other deficiencies in the art.

### SUMMARY OF THE INVENTION

The present invention relates to an optical trapping particle including a birefringent crystalline particle having a body and a length extending between a first end and a second end, said particle comprising an optic axis perpendicular to the length of the body, wherein the length of the body is greater than the largest width dimension of the first or second ends.

The present invention also relates to an optical trapping particle including an optically isotropic particle having a body and a length extending between a first end and a second end, said particle having an asymmetric cross-section, wherein the length of the body is from about 10 nanometers to about 10 micrometers and is greater than the largest width dimension of the first or second ends.

Another aspect of the present invention relates to an angular optical trap system. The system includes a sample chamber and an optical trapping particle. The optical trapping particle includes a birefringent crystalline particle having a body and a length extending between a first end and a second end, said particle comprising an optic axis perpendicular to the length of the body. The optical trapping particle is positioned within the sample chamber. The system also includes an angular optical trap assembly including a laser, a laser polarization rotator, and an input polarization detector, wherein the laser is positioned to generate an input trapping beam that passes through the laser polarization rotator to generate a first output trapping beam, wherein a first portion of the first output trapping beam passes into the input polarization detector and a second portion of the first output trapping beam passes into the sample chamber.

Yet another aspect of the present invention relates to an angular optical trap system. The system includes a sample chamber and an optical trapping particle. The optical trapping particle includes an optically isotropic particle having a body and a length extending between a first end and a second end, said particle having an asymmetric cross-section, wherein the length of the body is from about 10 nanometers to about 10 micrometers. The optical trapping particle is positioned within the sample chamber. The system also includes an angular optical trap assembly including a laser, a laser polarization rotator, and an input polarization detector, wherein the laser is positioned to generate an input trapping beam that passes through the laser polarization rotator to generate a first output trapping beam, wherein a first portion of the first output trapping beam passes into the input polarization detector and a second portion of the first output trapping beam passes into the sample chamber.

A further aspect of the present invention relates to a method of making a plurality of optical trapping particles. The method involves providing a birefringent crystalline wafer having a top surface and a bottom surface. Then, a plurality of substantially uniform post structures are formed within the wafer, wherein each post structure has a top end and a base end and wherein the base end is secured to the wafer. The substantially uniform post structures are released from the wafer to yield a plurality of substantially uniform optical trapping particles, wherein each particle has a body, a first end, and a second end.

Angular trapping and torque detection using optical trapping particles of the present invention is demonstrated. In the present invention, it is shown that the nanofabricated optical trapping particles allow direct and simultaneous measurement of torque, angle, force, and position with high resolution and bandwidth as demonstrated by measurements of DNA supercoiling described in the Examples below. In particular, the optical trapping particles of the present invention allow the confinement of all three degrees of rotational freedom in the systems of the present invention. The torque acting on the particle and its deviation from the trap direction are determined by direct measurement of the change in angular momentum of the transmitted beam. The ability to measure instantaneous torque is of great importance, since it facilitates precise measurement of the torque generated by biological structures as they rotate. The wide bandwidth and accuracy of the present systems allow the measurement of rotational motion of the trapped particle and to use feedback to control the applied torque or particle angle.

### BRIEF DESCRIPTION OF THE DRAWINGS

FIG. 1 is an illustration of an angular optical trapping configuration using a tapered cylinder. The tapered cylinder traps preferentially with the narrow side towards the microscope coverglass.

FIGS. 2A-B show an optically trapped cylindrical particle tethered at one edge to coverglass substrate. In FIG. 2A, the tether is relaxed and the cylinder orients vertically. In FIG. 2B, the tether is stretched out by the optical trap and the cylinder tips if the tether is not attached to the center of the cylinder end.

FIG. 3 is an illustration of an angular optical trapping configuration. A cylinder fabricated from pure crystalline quartz was designed to trap with its optic axis perpendicular to the propagation direction of the trapping laser. Its bottom surface was chemically functionalized for attachment to DNA. The height of the cylinder was greater than its diameter, causing the particle to align its cylinder axis with the laser propagation direction. The DNA was attached at one end to the bottom face of the cylinder via multiple biotin/streptavidin connections and at the other end to the surface of a coverglass via multiple digoxygenin/anti-digoxygenin connections. During a typical supercoiling experiment, the DNA was first stretched in the axial direction. The cylinder was then rotated via controlled rotation of the linear polarization of the laser to generate twist in the DNA.

FIG. 4 is a schematic representation of an angular optical trap system of the present invention. The inset shows the generation of DNA supercoiling using a quartz cylinder in an angular trap. The laser polarization rotator serves to rotate the polarization of the input trapping beam. The input angle detector (input polarization detector) determines the polarization angle of an output trapping beam from the polarization rotator. The torque/angle detector and force/position detector determine the torque, angular orientation, force, and position of the quartz particle.

FIG. 5 is a schematic of a DNA construct that includes attachment ends having a “T” shape for attachment to both an optical trapping particle and another substrate.

FIG. 6A shows a nanofabrication protocol for optical trapping particles. The quartz surface was cleaned and functionalized with 3-aminopropyltriethoxysilane (APTES). Photoresist was patterned onto the surface via projection lithography, and the patterned wafer was etched to create cylindrical posts. Residual photoresist was stripped from the posts by sonication in acetone to expose an active, functionalized surface. Posts were then removed with mechanical pressure from a microtome blade. FIGS. 6B-E show scanning electron micrographs of nanofabricated cylinders. FIGS. 6B and C show nanofabricated cylindrical posts on the wafer. The cylinders were 1.1 μm high and 0.53 μm in diameter. FIG. 6D shows the quartz substrate after a portion of the posts had been removed from the wafer. The quartz posts fractured evenly at their bases in a consistent manner. FIG. 6E shows a single quartz cylinder after mechanical removal. Scale bars indicate 5 μm in FIG. 6B and 1 μm in FIGS. 6C-E.

FIG. 7 shows light-driven micromotors of the present invention including propellers, drills, and polishers.

FIG. 8 shows the use of optical trapping particles of the present invention to study molecular motors, such as RNA polymerase.

FIGS. 9A-B show measurements during DNA supercoiling. A 2.2 kbp dsDNA molecule was held at 10 pN and positive supercoils were added at a rate of 2 turns/second. FIG. 9A shows the torque versus σ plot and FIG. 9B shows the corresponding extension versus σ plot.

FIG. 10 shows the experimental configuration for observing plectoneme formation in individual DNA molecules. A DNA molecule was tethered to a nanofabricated birefringent quartz cylinder (with χₑ being the electric susceptibility along the extraordinary (optic) axis) held in an angular optical trap. Both ends of the DNA were torsionally constrained via its multiple tags: at one end via biotin-streptavidin and at the other end via digoxigenin (dig) and anti-dig. Force on the cylinder was applied in the axial direction and held constant by feeding back on a piezoelectric stage which displaced the coverslip. The DNA molecule was subsequently overwound by rotation of the linear polarization of the trapping laser.

FIGS. 11A-B are graphs showing examples of torque and extension versus turn number. DNA molecules of 2.2 kbp in length were overwound under a constant force. Data were collected at 2 kHz and averaged with a sliding window of 1.5 seconds for torque and 0.05 seconds for extension. DNA buckling, locations indicated by dashed lines, was dependent on the applied force. FIG. 11A shows torque versus number of turns. FIG. 11B shows the corresponding extension versus number of turns.

FIGS. 12A-B are graphs showing extension change at the buckling transition. FIG. 12A shows the extension change versus force for initial and subsequent plectoneme formation. The extension change for the initial plectoneme formation (symbols on curve labeled buckling transition) was much larger and was well fit by a power law of F^{-0.5} (curve labeled buckling transition); whereas the extension change per turn for subsequent plectoneme formation (symbols labeled post-buckling per turn) was well fit by a power law of F^{-0.4} (fit not shown). The three different DNA templates used here (2.2 kbp , N=119; 4.2 kbp ⋄, N=35, and another 2.2 kbp ★, N=4) all exhibited the same trend. The two dashed lines show predictions by a simple model and a fit to the Marko model respectively. Error bars are standard errors of the means. In FIG. 12B, a molecule was held at constant tension (2 pN) and overwound extremely slowly (0.04 turn/second) through its buckling transition. Data were taken at 2 kHz, low pass filtered to 400 Hz (solid dots), and then median filtered to 20 Hz (curve). An extension histogram of the median-filtered data is shown on the right and was well fit by the sum of two Gaussians. The DNA was observed to rapidly fluctuate between two distinct states, separated by 79 nm, corresponding to pre- and post-plectonemic formation of the first loop.

FIGS. 13A-B are graphs showing direct measurements of torque. FIG. 13A shows torque prior to the buckling. Torque-turn relations prior to buckling were pooled from data for both the 2.2 kbp DNA (121 traces) and 4.2 kbp DNA (65 traces). Individual traces are shown as grey lines and resulted in a grey region when plotted together. Each solid curve indicates the average of all traces of a given length DNA. Torsional modulus for each DNA length (see Examples for values) was obtained by the slope of a linear fit to the average curve. FIG. 13B shows torque after buckling. The torque after buckling at each force was pooled from data from the three DNA templates (blue symbols; 2.2 kbp , N=119; 4.2 kbp ⋄, N=35; and another 2.2 kbp ★, N=4). The two dashed lines show predictions by the simple model and a fit to the Marko model, respectively.

FIGS. 14A-J show an optimized protocol for forming cylindrical quartz optical trapping particles.

FIG. 15 is a picture of cylindrical quartz trapping particles with resist on top appearing after anisotropic dry etch was performed.

FIG. 16 is a picture of cylindrical quartz trapping particles where an isotropic etch of photoresist was just executed.

FIG. 17 shows cylindrical quartz trapping particles where the photoresist was removed using acetone. There is a 10 nm step on the quartz cylinders; this was done in order to assure that the area not covered by resist was not functionalized.

### DETAILED DESCRIPTION OF THE INVENTION

The present invention relates to an optical trapping particle including a birefringent crystalline particle having a body and a length extending between a first end and a second end, said particle comprising an optic (extraordinary) axis perpendicular to the length of the body, wherein the length of the body is greater than the largest width dimension of the first or second ends.

As used herein, birefringent crystalline particles are anisotropic materials which refract light in two different ways to form two rays. The particle may comprise any birefringent material, natural or synthetic, including, but not limited to quartz, sapphire, mica, calcite, corundum, beryl, rutile, tourmaline, calomel, lithium niobate, magnesium fluoride, ruby, peridot, zircon, topaz, olivine, perovskite, and nepheline. In a preferred embodiment, the birefringent material is a positive crystal, such as quartz.

In addition, the particle may have any desired cross-sectional shape. Suitable cross-sectional shapes include, but are not limited to, circular, elliptical, or polygonal cross-sectional shapes. As used herein, a polygonal cross-sectional shape includes, but is not limited to, a triangle, a square, a trapezoid, a rectangle, a parallelogram, a pentagon, a hexagon, a star shape, and a polygon having seven or more sides (including, for example, a gear shape). In a preferred embodiment, the particle is cylindrical, having a circular cross-section.

In one embodiment of the present invention, the first or second ends are capable of coupling to a target molecule or attachment device. In one preferred embodiment of the present invention, at least a portion of the first or second ends, preferably the second end, includes a functional group capable of coupling to a target molecule or attachment device. Suitable target molecules include, but are not limited to, a nucleic acid molecule, a protein molecule, a polypeptide, or an organic polymer. Suitable nucleic acid molecules include, but are not limited to, ribonucleotides, deoxyribonucleotides, modified ribonucleotides, modified deoxyribonucleotides, peptide nucleic acids, modified peptide nucleotide analogues, modified phosphate-sugar backbone oligonucleotides, nucleotide analogues, or mixtures thereof. The target molecule can be coupled to the optical trapping particle via any desired coupling mechanism including, but not limited to, covalent bonding, non-covalent bonding, ionic bonding, hydrogen bonding, van der Waals interactions, and the like.

In another embodiment, the first or second ends of the optical trapping particle include one or more functional groups capable of attaching to an attachment device. In a preferred embodiment, the attachment device is a propeller, drill, polisher, grinder, mill, or gear. In this embodiment, the optical trapping particle can serve as a microscopic machine, such as a light-driven micromotor. For example, the optical trapping particle can be specifically attached to a propeller-type structure by taking advantage of the particle's single functionalized surface. The propeller or other microstructures may be driven by rotating the particle with light energy. The optical trapping particle can be pressed against a surface and rotated. Different attachment devices can be placed on the functionalized particle end and used to drill or polish very specific and small locations with great precision. The ability to place attachment devices on a specific end of the particle allows the user to generate just about any rotationally driven tool imaginable. The optical trapping particle acts as the “motor”, and the attachment device can be coupled to the motor via any desired coupling mechanism including, but not limited to, covalent bonding, non-covalent bonding, ionic bonding, hydrogen bonding, van der Waals interactions, and the like.

The desired functional groups for the second end of the optical trapping particle will be determined by the target molecule or attachment device to be used in conjunction with the optical trapping particle and can be readily determined by one of ordinary skill in the art. Suitable functional groups for the second end include, but are not limited to, olefin, amino (e.g., APTES), thiol (e.g., SPDP), hydroxyl, silanol, aldehyde, keto, halo, acyl halide, or carboxyl groups.

In one preferred embodiment, only a selected region (e.g., a center portion) of the second end is functionalized for coupling to the target molecule or attachment device. In this embodiment, the functionalized area is reduced to prevent the unwanted precessing of a cylinder in an optical trap, thereby minimizing measurement noise and giving more accurate measurements.

In another embodiment of the present invention, the optical trapping particle may include a belt, chain, or string. As used herein, a belt, chain, or string can be wrapped around the particle and used to couple its motion to another device.

In another preferred embodiment, the length of the body is between about 10 nanometers and about 10 micrometers. In yet another preferred embodiment, the largest width dimension of the first or second ends is less than 10 micrometers.

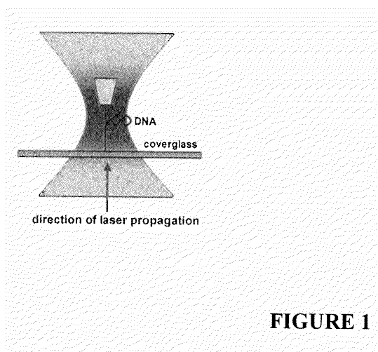

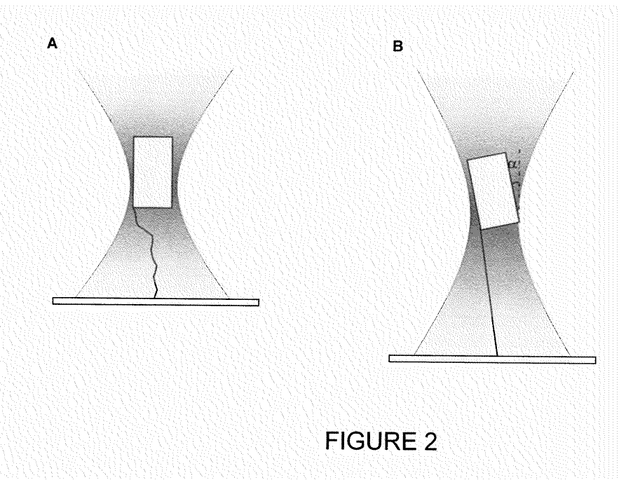

In one embodiment of the present invention, the surface area of the first and second ends of the particle is the same. In another embodiment, the body of the particle is tapered such that the surface area of one end is larger than the surface area of the other end. In a preferred embodiment, the body is tapered from the first end to the second end such that the surface area of the first end is larger than the surface area of the second end. The degree of tapering may be controlled during the fabrication process and may range from about a zero degree inclination to about a 25 degree inclination, resulting in a region at the second end of at least a few nanometers. When such a tapered particle is trapped in an angular optical trap, it orients its narrow end upstream of the direction of trapping laser propagation and facing the coverglass and objective (FIG. 1). This has two major advantages. First, since the narrow end is functionalized, the tapered particle naturally assumes the correct orientation when a molecule of interest is attached to both the particle and a microscope coverglass as shown in FIG. 1. Second, tapering the end decreases the available area for target molecule attachment, and the target molecule is more likely to bind near the center of the particle end, thereby minimizing the possibility of particle tipping when the attached molecule is stretched axially (see FIG. 2).

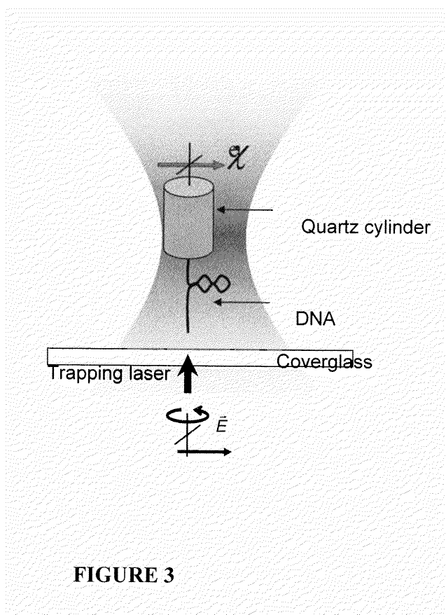

Ideally, optical trapping particles used to generate both torque and force should have the following attributes: (1) optical anisotropy for generation of torques well suited for biological applications, (2) confinement of all three rotational degrees of freedom to achieve a true angular trap, (3) specific chemical derivatization at a well-defined location on the handle for attachment to a molecule of interest, (4) independent control of the application of force and torque, and (5) uniform size, shape, and optical properties for ease of calibration and reproducibility. The above attributes are possessed by the optical trapping particles of the present invention. In particular, the above attributes are possessed by a particle with its optic axis perpendicular to the length of the body and one of its ends chemically derivatized (FIG. 3). Since an optical trapping particle, e.g., a cylinder, with a height greater than its largest width dimension will have a tendency to align with the laser propagation direction, provided that the largest width dimension is comparable to the beam diameter (Singer et al., Phys. Rev. E, 73:021911 (5 pages) (2006); Ashkin et al., Nature 330:769-771 (1987), which are hereby incorporated by reference in their entirety), an optical trapping particle with its optic axis perpendicular to the long axis can be rotated about its long axis by rotation of the linear polarization of the trapping laser. In this design, the optical anisotropy confines two of the three rotational degrees of freedom, while the shape anisotropy also confines the third degree of freedom to achieve a true angular trap. Attachment of a biological molecule to one end of an optical trapping particle allows the application of force to the molecule along the laser propagation direction without significant tilting of the trapped particle. This ensures that the optic axis is maintained perpendicular to the laser propagation direction even when the attached molecule is under tension. This is desirable because tilting of the cylinder would result in suboptimal application of torque, and loss of independent control of torque and force. Nanofabrication techniques (described below) allow for the mass production of particles of uniform size, shape, and optical properties as well as specific chemical derivatization of only one end of each particle.

The present invention also relates to an optical trapping particle including an optically isotropic particle having a body and a length extending between a first end and a second end, said particle having an asymmetric cross-section, wherein the length of the body is from about 10 nanometers to about 10 micrometers and is greater than the largest width dimension of the first or second ends.

As used herein, optically isotropic but asymmetric particles exhibit a stronger optical polarizability when measured along one of the axes perpendicular to the length of the body, due to optical shape anisotropy. Suitable materials for the optically isotropic particle include, but are not limited to, glass, silicon, plastics, such as polystyrene, fused silica, fused quartz, pyrex, BK7, and silica.

In a preferred embodiment, the particle has an asymmetric cross-sectional shape selected from the group consisting of elliptical and asymmetric polygons. As used herein, an asymmetric cross-sectional shape has a long axis and at least one shorter axis. The asymmetric cross-sectional shape allows the long axis of the cross-section to be oriented with the E field of a trapping beam.

Another aspect of the present invention relates to an angular optical trap system. The system includes a sample chamber and an optical trapping particle. The optical trapping particle includes a birefringent crystalline particle having a body and a length extending between a first end and a second end, said particle comprising an optic axis perpendicular to the length of the body. The optical trapping particle is positioned within the sample chamber. The system also includes an angular optical trap assembly including a laser, a laser polarization rotator, and an input polarization detector, wherein the laser is positioned to generate an input trapping beam that passes through the laser polarization rotator to generate a first output trapping beam, wherein a first portion of the first output trapping beam passes into the input polarization detector and a second portion of the first output trapping beam passes into the sample chamber.

Yet another aspect of the present invention relates to an angular optical trap system. The system includes a sample chamber and an optical trapping particle. The optical trapping particle includes an optically isotropic particle having a body and a length extending between a first end and a second end, said particle having an asymmetric cross-section, wherein the length of the body is from about 10 nanometers to about 10 micrometers. The optical trapping particle is positioned within the sample chamber. The system also includes an angular optical trap assembly including a laser, a laser polarization rotator, and an input polarization detector, wherein the laser is positioned to generate an input trapping beam that passes through the laser polarization rotator to generate a first output trapping beam, wherein a first portion of the first output trapping beam passes through the input polarization detector and a second portion of the first output trapping beam passes into the sample chamber.

In one embodiment of the present invention, the length of the body of the optical trapping particle is greater than the largest width dimension of the first or second ends. In another embodiment, the length of the body of the optical trapping particle is smaller than the largest width dimension of the first or second ends. In this embodiment, the optical trapping particle is an a disk-like formation, with any desired cross-sectional shape as described above. Moreover, in this embodiment, the disk is oriented on its edge in the angular optical trap system and rotates about an axis parallel to the ends. In particular, for an optically isotropic particle having an asymmetric cross-section, the long axis of the disk can align with the polarization of the electric field. In the embodiment including a birefringent crystalline particle, since the disk is oriented on its edge in the trap, the additional alignment of the optic axis with the electric field will prevent rotations of the disk about is length-wise axis. Therefore, all three degrees of rotation are confined.

The polarization of the laser can then be rotated (i.e., rotation of the E field) to spin the disk about an axis along the direction of laser propagation.

Angular optical trap assemblies suitable for use in the present invention are known in the art and are described, for example, in La Porta et al., “Optical Torque Wrench: Angular Trapping, Rotation, and Torque Detection of Quartz Microparticles,” Physical Review Letters, 92:190801 (4 pages) (2004), which is hereby incorporated by reference in its entirety.

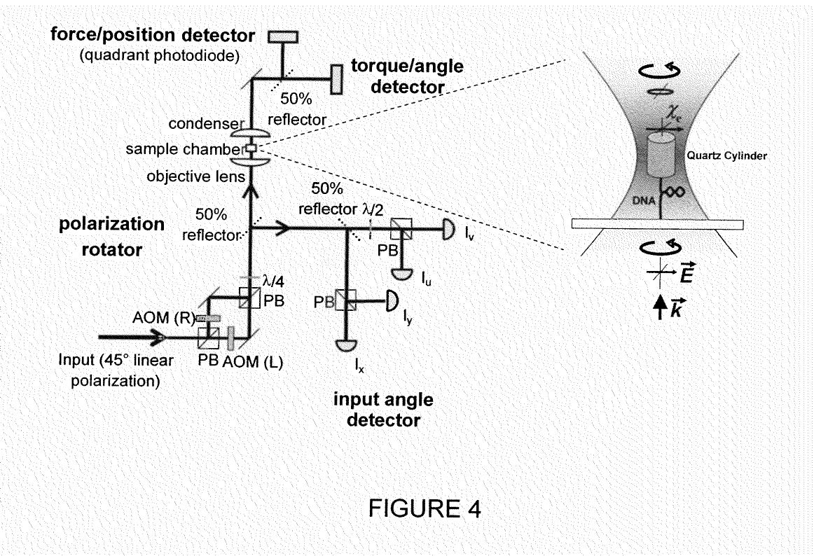

One embodiment of an angular optical trap system including an angular optical trap assembly is shown in FIG. 4. In particular, the optical trap assembly includes a laser. In accordance with the present invention, any suitable laser, continuous wave or pulsed, IR or other wavelength, with a well defined polarization can be used. In a preferred embodiment, the laser is a continuous wave IR laser of a well defined linear polarization.

The laser generates an input trapping beam which travels into the laser polarization rotator. As shown in FIG. 4, in the laser polarization rotator, acousto-optic modulators (AOMs) marked L and R generate the left and right circular polarization components of the output beam, and the relative phase of these components is determined by the relative phase of the AOM radiofrequency (rf) drives. As a result, a relative phase shift φ of the rf signals causes a rotation of φ/2 of the output polarization. The AOM drive signals are generated by computer-controlled digital frequency synthesis, allowing the polarization angle to be changed with a response time of a few microseconds. The first polarization beam (PB) splitting cube splits the laser into two beams of orthogonal polarizations, which then enter into the two AOMs. The second polarization beam splitting cube subsequently recombines the two polarizations after they have passed through the AOMs. Although the use of multiple AOMs is shown in FIG. 4, the laser polarization rotator may include any device capable of rotating polarization of the laser beam. For example, mechanical rotation of optical elements can be used (e.g., by hand or any mechanical device). An alternative embodiment involves the use of an electro-optic modulator that rotates the polarization of the input beam. The input polarization does not have to be linear and can be circular or elliptical polarizations.

In accordance with the present invention, a fraction of the output laser trapping beam is deflected into the laser input polarization (angle) detector, which detects both the polarization angle of the laser beam as well as its ellipticity. The remaining fraction of the output laser beam then enters into the sample chamber via an objective lens of a microscope to trap the optical trapping particle before existing from the condenser. Both the objective and condenser lenses can be those from a conventional research-grade microscope with sufficiently high numerical apertures (typically >0.9) suitable for trapping and detection.

In the embodiment shown in FIG. 4, after the laser interacts with the trapped particle, it is split into two paths. One path enters the torque/angle detector that separates the left and right circular polarization components of the beam and sends each component to one of two photodetectors. The intensity imbalance between the two beams is used to detect the torque exerted on the particle and the angle of polarization. The other path enters a force/position detector that comprises a quadrant photodetector and that detects the force exerted on the particle and the three-dimensional location of the trapped particle within the laser beam. Although two types of detectors are shown in FIG. 4, the optical trap assembly may include none or any number of desired detectors. The angular orientation and the position of the particle may also be detected via direct imaging of the particle at the specimen (e.g., using a camera).

The angular optical trapping assembly of the present invention, described in detail previously (La Porta et al., Phys. Rev. Lett., 92:190801 (4 pages) (2004), which is hereby incorporated by reference in its entirety) features precise and immediate control of the trapping beam's linear polarization, which is used to rotate a trapped optical trapping particle about its long axis. The physical torque exerted on the particle is determined by direct measurement of the change in angular momentum of the transmitted beam (La Porta et al., Phys. Rev. Lett., 92:190801 (4 pages) (2004), which is hereby incorporated by reference in its entirety).

In a preferred embodiment, as shown in the inset of FIG. 4, the angular optical trap system includes an optical trapping particle complex. The optical trapping particle complex includes the optical trapping particle and a target molecule or attachment device attached at a first position to the second end of the optical trapping particle to form the complex. In this embodiment shown in FIG. 4, the complex includes a target molecule (e.g., DNA). The complex is positioned within the sample chamber of the angular optical trap system.

In yet another embodiment, as shown in FIG. 4, the optical trapping particle complex comprises a target molecule (e.g., DNA) and further comprises a substrate, wherein the target molecule is attached at a second position to the substrate. In this embodiment, the substrate may be translated in three dimensions and allows stretching of the target molecule through the optically trapped particle. Suitable substrates include, but are not limited to, glass coverslips, glass plates, plastic coverslips, plastic plates, or any other substrates compatible with optical trapping.

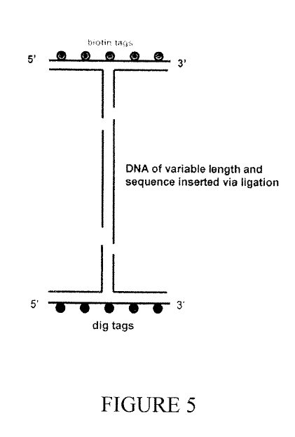

In another preferred embodiment of the present invention, at least one of the first and second positions of the target molecule includes a T-shaped portion suitable for attaching to the optical trapping particle or substrate. This is shown, for example, in FIG. 5. In this embodiment, a DNA construct (i.e., target molecule) has attachment ends that have a “T” shape. One “T” end binds to the second end of the optical trapping particle and the other “T” end binds to a surface of a coverglass substrate. The T ends have the advantage that they are less likely to tilt the optical trapping particle when the DNA molecule is stretched. Linear DNA has a persistence length of approximately 50 nm. Therefore, in order for a linear DNA to make a sharp 90 degree turn at the particle surface, torque will be exerted on the DNA by the particle, resulting in particle-tipping when the DNA is pulled taut. The DNA with T-ends should avoid this problem.

The angular trap is based on the fact that a dielectric material subject to an external electric field E (constant or oscillating) generates polarization P given by P=χE, where χ is the electric susceptibility. If the material is birefringent, the susceptibility is not isotropic so that the expression for the polarization is generalized to P=χₓEₓ\hat{x}+χ_{y}E_{y}ŷ+χ_{z}E_{z}\hat{z}, where \hat{x}, ŷ and \hat{z} are unit vectors along the principal axes of the crystal and χₓ, χ_{y} and χ_{z} are the corresponding electrical susceptibilities (Yariv, Optical Electronics, Holt, Rinehart, and Winston, New York (1985), which is hereby incorporated by reference in its entirety). For typical uniaxial birefringent materials such as quartz or calcite, two of the susceptibilities are equal (χₒ ordinary) and the third is different (χₑ extraordinary).

In one embodiment, angular trapping occurs in particles made from birefringent materials, in which the optic axis of the crystal is more easily polarized than the ordinary axes. In this case the polarization P induced on a particle by an external electric field E will be tilted toward the optic axis. The misalignment between E and P results in a torque given by

$$τ = \int {d}^{3} x P \times E = \hat{q} \frac{1}{2} ( {χ}_{o} - {χ}_{e} ) sin 2 θ \int {d}^{3} x {E}_{0}^{2} ( x ) = \hat{q} {τ}_{0} sin 2 θ \qquad ( 1 )$$

where θ is the angle between E and the optic axis, \hat{q} is a unit vector perpendicular to E and P, and τ₀ is the maximum magnitude of torque that can be exerted on the particle. (Particle shape effects are neglected in this formula.) As a result, linearly polarized light can be used to exert torque on an optical trapping particle. This torque tends to align the optic axis of the particle with the electric field direction, as shown FIG. 4.

In order to detect the torque, the conservation of angular momentum, which requires that the torque acting on the particle is equal and opposite to the rate of change of the angular momentum of the trapping beam as it passes through the particle, is taken advantage of. Since the torque is generated using polarization properties, the angular momentum is transferred to the polarization state of the transmitted beam rather than to its spatial profile. Light with left (right) handed circular polarization contains angular momentum +(−) and energy ωₒ per photon, where is the reduced Planck constant and ωₒ is the optical angular frequency. The linearly polarized trap beam contains no net angular momentum because it is composed of equal quantities of left and right circular polarization. Exertion of torque τ on a particle causes an imbalance of the power of left and right circular components (P_{L} and P_{R}) in the transmitted beam, such that τ=(P_{R}−P_{L})/ω₀. Direct measurement of this quantity is made by the torque detector shown in FIG. 4. In principle, the torque is strictly determined by the angular momentum content of the transmitted beam (Nieminen et al., Journal of Modern Optics, 48:405-413 (2001); Bishop et al., Physical Review A, 68:033802 (8 pages) (2003), which are hereby incorporated by reference in their entirety). In practice, it is impossible to collect the transmitted trap beam in its entirety, so a calibration of the detector is necessary.

The first step in the calibration procedure is to relate the torque signal to the deviation of the particle from the trap polarization angle. Referring to Equation 1, the angle is given by θ=(1/2)arcsin(V_{τ}/V₀), where V_{τ} is the torque signal in volts and V₀ is the maximum value of this signal, obtained at θ=45°. The value of V₀ may be determined by rotating the polarization much faster than the particle can follow, so that the polarization vector scans the quasi-stationary particle. The amplitude of the resulting sinusoidal modulation is V₀. For small angles it can be approximated as θ≈V_{τ}/2V₀.

Once the angular calibration is accomplished, angular deviation can be determined from the torque signal. The task remains to determine the stiffness of the angular trap and convert the torque signal to physical units of torque. Applying the standard treatment of Brownian fluctuations in a potential well to rotational motion, it is found that the power spectral density of the angular fluctuations is of the form S(f)=A²/(f²+f₀²) with corner frequency f₀=κ/2πξ and amplitude A²=k_{B}T/ξπ², where k_{B} is the Boltzmann constant, T is the temperature in degrees kelvin, κ is the stiffness of the angular trap, and ξ is the rotational viscous damping coefficient. The damping ξ and stiffness κ care determined by fitting the predicted function to the measured power spectrum. Once the angular trap stiffness is known the torque is related to the raw torque signal by τ=V_{τ}(κ/2V₀). The torque sensitivity obtained from the calibration is within experimental error of the absolute angular momentum change of the trap beam, taking into account our estimated ˜50% light collection efficiency.

The calibration of torque allows the direct measurement of the viscous drag on a spinning particle as a function of rotation rate.

Although FIG. 4 shows the use of a single angular optical trap, extension to multiple angular traps is also possible. For example, multiple traps may be generated by time-sharing the trapping laser at different locations at a rate much faster than the corner frequency of trapped optical trapping particles (Molloy et al., Nature, 378(6553):209-212 (1995), which is hereby incorporated by reference in its entirety). This also allows the location and polarization of each trap independently controlled. Using fast detectors, the force, position, torque, and angular orientation for each trapped particle may also be determined in real time. Alternatively, multiple traps may also be generated by sending the trapping laser through spatial light modulators or addressable and steerable mirror arrays, which are capable of creating multiple laser beams from a single beam. Force, position, torque, and angular orientation for trapped particles may be simultaneously detected by an array of detectors including cameras.

Another aspect of the present invention relates to a method of making one or more substantially uniform optical trapping particles. This method involves providing a birefringent crystalline wafer having a top surface and a bottom surface. Then one or more substantially uniform post structures are formed within the wafer, wherein each post structure has a top end and a base end and wherein the base end is secured to the wafer.

The one or more substantially uniform post structures are released from the wafer to yield one or more substantially uniform optical trapping particles, wherein each particle has a body, a first end, and a second end.

The post structures can have any desired cross-sectional shape, as described above. In one embodiment, the length of the body measured from the first end to the second end is greater than the largest width dimension of the first or second ends. In another embodiment, the length of the body measured from the first end to the second end is smaller than the largest width dimension of the first or second ends (e.g., a disk).

In one preferred embodiment, the length of the body is between about 10 nanometers and about 10 micrometers. In yet another preferred embodiment, the largest width dimension of the first or second ends is less than about 10 micrometers. In a further embodiment, the top surface and the bottom surface each have a surface area of between about 6 square centimeters and about 300 square centimeters.

The post structures can be formed using techniques known to those of ordinary skill in the art. In one embodiment, forming involves using optical lithography to form the plurality of substantially uniform post structures within the wafer. Some of these procedures for nanofabricating posts are similar to those previously described (Volkmuth et al., Nature, 358:600-602 (1992), which is hereby incorporated by reference in its entirety). In another embodiment, forming involves using electron beam lithography to form the plurality of substantially uniform post structures within the wafer.

In yet another embodiment, forming involves using holographic lithography to form the plurality of substantially uniform post structures within the wafer (Sharp et al., “Photonic Crystals for the Visible Spectrum by Holographic Lithography,” Optical and Quantum Electronics, 34(1-3): 3-12 (2002); Turberfield, “Photonic Crystals Made by Holographic Lithography,” MRS Bulletin, 26(8):632-636 (2001), which are hereby incorporated by reference in their entirety).

Releasing can be achieved using mechanical pressure to separate the base end of the post-like nanostructures from the wafer. Suitable techniques for using mechanical pressure to separate the post-like structures include, but are not limited to, pressure with a microtome blade. In another embodiment, releasing is achieved through the use of a liftoff (or sacrificial) layer. In particular, in this embodiment, a liftoff layer is applied to the top surface of a substrate and the birefringent crystalline wafer is positioned adjacent a top surface of the liftoff layer prior to formation of the post structures. After formation of the post structures, the liftoff layer is chemically removed (e.g., with a solvent) and the post structures are released. Suitable liftoff layers and techniques for chemically removing the liftoff layer will be determined by the liftoff layer used and can be readily determined by one of ordinary skill in the art.

In one embodiment, the method further involves functionalizing at least a portion of the top surface of the wafer prior to the deposition of the photoresist during nanofabrication so that the functionalized top surface is capable of coupling to a target molecule or attachment device. In an alternative embodiment, the method further involves functionalizing at least a portion of the top end of each of the post structures so that the functionalized top end is capable of coupling to a target molecule or attachment device.

The top surface of the wafer or at least a portion of the top end of the post structure can be functionalized with any desired functional group including, but not limited to, olefin, amino, thiol, hydroxyl, silanol, aldehyde, keto, halo, acyl halide, or carboxyl groups. Wafer surfaces may be functionalized for biomolecule attachment using standard techniques (for coupling to an amine group, see Kleinfeld et al., J. Neurosci., 8:4098-4120 (1988), which is hereby incorporated by reference in its entirety).

In one embodiment, the top surface of the wafer or at least a portion of the top end of the post structure is functionalized with an amino group by reaction with an amine compound selected from the group consisting of 3-aminopropyl triethoxysilane, 3-aminopropylmethyldiethoxysilane, 3-aminopropyl dimethylethoxysilane, 3-aminopropyl trimethoxysilane, N-(2-aminoethyl)-3-aminopropylmethyl dimethoxysilane, N-(2-aminoethyl-3-aminopropyl)trimethoxysilane, aminophenyl trimethoxysilane, 4-aminobutyldimethyl methoxysilane, 4-aminobutyl triethoxysilane, aminoethylaminomethylphenethyl trimethoxysilane, and mixtures thereof.

In another embodiment, the top surface of the wafer or at least a portion of the top end of the post structure is functionalized with an olefin-containing silane. In this embodiment, the olefin-containing silane is selected from the group consisting of 3-(trimethoxysilyl)propyl methacrylate, N-[3-(trimethoxysilyl)propyl]-N′-(4-vinylbenzyl)ethylenediamine, triethoxyvinylsilane, triethylvinylsilane, vinyltrichlorosilane, vinyltrimethoxysilane, vinyltrimethylsilane, and mixtures thereof.

In yet another embodiment, the top surface of the wafer or at least a portion of the top end of the post structure is polymerized with an olefin containing monomer. In a preferred embodiment, the olefin-containing monomer contains a functional group. Suitable olefin-containing monomers include, but are not limited to, acrylic acid, methacrylic acid, vinylacetic acid, 4-vinylbenzoic acid, itaconic acid, allyl amine, allylethylamine, 4-aminostyrene, 2-aminoethyl methacrylate, acryloyl chloride, methacryloyl chloride, chlorostyrene, dichlorostyrene, 4-hydroxystyrene, hydroxymethylstyrene, vinylbenzyl alcohol, allyl alcohol, 2-hydroxyethyl methacrylate, poly(ethylene glycol) methacrylate, and mixtures thereof.

In a further embodiment, the first end and/or second end of the particle is polymerized with a monomer selected from the group consisting of acrylic acid, acrylamide, methacrylic acid, vinylacetic acid, 4-vinylbenzoic acid, itaconic acid, allyl amine, allylethylamine, 4-aminostyrene, 2-aminoethyl methacrylate, acryloyl chloride, methacryloyl chloride, chlorostyrene, dichlorostyrene, 4-hydroxystyrene, hydroxymethyl styrene, vinylbenzyl alcohol, allyl alcohol, 2-hydroxyethyl methacrylate, poly(ethylene glycol) methacrylate, and mixtures thereof, together with a monomer selected from the group consisting of acrylic acid, methacrylic acid, vinylacetic acid, 4-vinylbenzoic acid, itaconic acid, allyl amine, allylethylamine, 4-aminostyrene, 2-aminoethyl methacrylate, acryloyl chloride, methacryloyl chloride, chlorostyrene, dichlorostyrene, 4-hydroxystyrene, hydroxymethyl styrene, vinylbenzyl alcohol, allyl alcohol, 2-hydroxyethyl methacrylate, poly(ethylene glycol) methacrylate, methyl acrylate, methyl methacrylate, ethyl acrylate, ethyl methacrylate, styrene, 1-vinylimidazole, 2-vinylpyridine, 4-vinylpyridine, divinylbenzene, ethylene glycol dimethacrylate, N,N′-methylenediacrylamide, N,N′-phenylenediacrylamide, 3,5-bis(acryloylamido) benzoic acid, pentaerythritol triacrylate, trimethylolpropane trimethacrylate, pentaerytrithol tetraacrylate, trimethylolpropane ethoxylate (14/3 EO/OH) triacrylate, trimethylolpropane ethoxylate (7/3 EO/OH) triacrylate, trimethylolpropane propoxylate (1 PO/OH) triacrylate, trimethylolpropane propoxylate (2 PO/OH) triacrylate, and mixtures thereof.

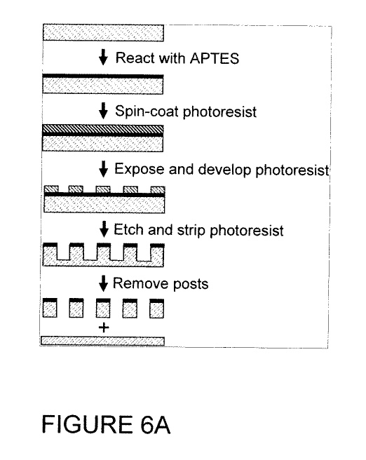

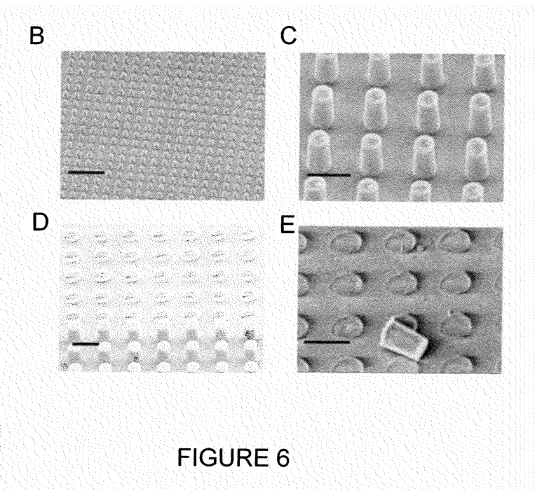

One embodiment of the method for making optical trapping particles is shown in FIG. 6A. In particular, the top surface of the crystalline wafer is functionalized with 3-aminopropyltriethoxysilane (APTES). Then a photoresist was spun onto the top surface of the wafer, patterned by optical lithography, and subsequently developed. The patterned wafer was then dry-etched, e.g., a plasma oxide etch, taking care so that the photoresist was not completely etched away, resulting in the formation of a plurality of substantially uniform post structures which are capped by the photoresist layer. The photoresist also protects the underlying functional groups which may be damaged during the etching step. The photoresist caps are then stripped using, for example, a solvent that will not damage the functional groups. This results in post structures in the wafer having a functionalized top end. The post structures are then removed from the wafer as described above. Additional functionalization can then be performed to obtain desired functional groups on the first or second ends of the optical trapping particles. Material processing techniques may be employed to make the particles more regular in shape (Sun et al., Sensors and Actuators B, 13:107-110 (1993), which is hereby incorporated by reference in its entirety).

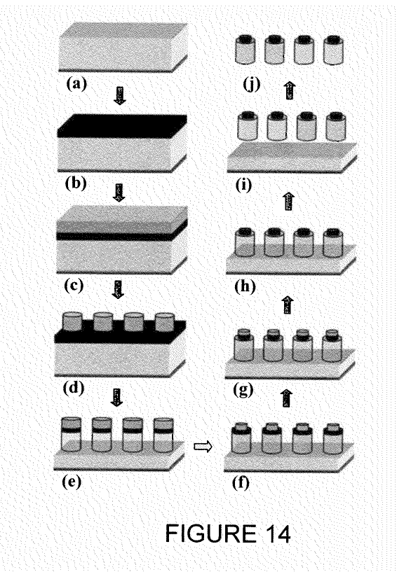

As described above, in one embodiment of the present invention only a selected region (e.g., a center region) of the second end of the optical trapping particle includes one or more functional groups capable of attachment to a target molecule or attachment device. This can be achieved by first functionalizing the entire surface of the wafer, and then using photolithography and oxygen plasma to selectively destroy the functionalization of any undesired regions. An alternate method involves processing the pillars or posts just before residual photoresist is sonicated off to expose the amine groups. Various methods might be employed to remove photoresist only near the edges of the particle, while retaining photoresist near the center. Example 5 and FIGS. 14-17 show one method of reduction in the functionalized area. Here an isotropic dry etch is performed in which the resist is, for the most, symmetrically etched away. A second anisotropic etch is performed in section and this creates a 10 nm step in the quartz and provides assurance that all of the unprotected APTES is removed. Instead of the second anisotropic etch, the functionalization on particle regions not covered by photoresist can be blocked chemically. Thus, particles can be formed that are only functionalized very close to the particle's long axis, and the molecule of interest would only bind in a small region in the center as is desired. Finally, a very small functionalized region (−10 nm) may be directly achieved using E-beam lithography.

The optical trapping particles are nanofabricated to ensure uniformity and thus are ideally suited for calibration and measurement reproducibility. Each particle is also chemically functionalized on one end for specific attachment to DNA.

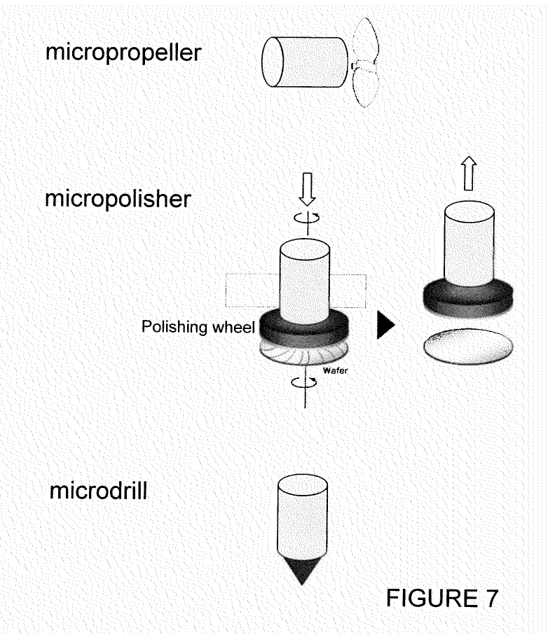

The optical trapping particles and systems of the present invention have many uses. In particular, the particles can be viewed as small stirring rods that allow for precise mixing of very small volumes, as small as femtoliters. In addition, the particle can be used to open and close a microvalve. It can also be used as a microspool to wrap polymer (such as DNA) around. Moreover, the particle can be used as part of a light-driven micromotor, micropolisher, or microdrill, as described above. Examples of such microdevices are shown in FIG. 7.

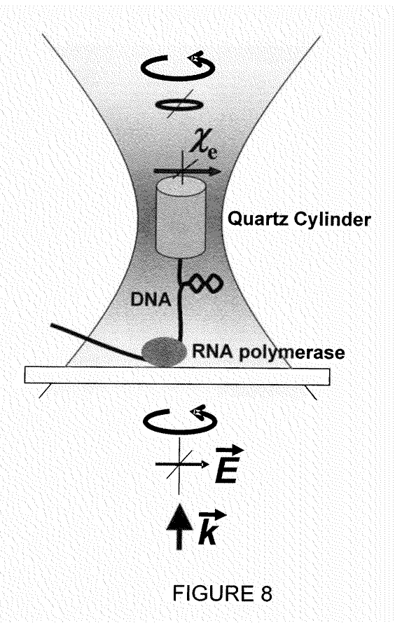

In another embodiment, the angular optical trap systems can be used to study molecular motors, such as RNA polymerase. Many enzymes involved in DNA replication, DNA recombination and repair, and RNA synthesis may track along the groove of the DNA double helix. Such groove tracking enzymes will apply torsional stress to the DNA and exhibit rotational motion as they translate. This is shown, for example, in FIG. 8, where an RNA polymerase motor is torsionally constrained to the surface of a glass coverslip. During transcription, RNA polymerase translocates along the DNA while rotating around the helical axis of the DNA and thus generating torsion on the optical trapping particle. This configuration allows the study of RNA polymerase under torsion, especially DNA supercoiling. Similarly, the behavior of DNA-binding proteins that bend and twist DNA might be studied using the system of the present invention. This system is also ideally suited to monitor the rotational motion of other torque-generating enzymes including, but not limited to, bacterial flagella moor, F1-ATPase, and dynein.

Molecular motors are known to generate torque ranging from tens to thousands of pN·nm (Noji et al., Nature, 386:299-302 (1997); Ryu et al., Nature, 403:444-447 (2000), which are hereby incorporated by reference in their entirety), which is well within the dynamic range of the system without requiring excessive trap power. Most importantly, the instantaneous readout and feedback capabilities will allow the torque generated by a biological structure in response to the imposed rotation (or vice versa) to be continuously measured.

The systems of the present invention are also ideally suited to investigate the torsional properties of biopolymers. In particular, as described below in the Examples, they can be used to probe DNA supercoiling dynamics. During DNA supercoiling, torque, angle, force, and extension of a DNA molecule can be simultaneously monitored at kHz rates using the systems of the present invention. In accordance with the present invention, the torsional modulus of DNA in the intermediate force regime can be directly measured, basic relations regarding the dependence of torque on applied force can be determined, the abrupt formation of the initial plectoneme in overwound DNA was first observed (see Examples below), and previously unseen dynamics of plectoneme formation can be monitored.

The rotational motions of a trapped optical trapping particle are sensitive to viscosity and proximity to other surfaces at a microscopic scale. Thus, analysis of these rotational motions may serve as a method to measure local viscosity.

The systems described herein have the important advantage that angular trapping is combined with detectors allowing instantaneous measurement of the torque acting on the particle and its angular deviation from the trap direction in addition to the force exerting on the particle and the position of the particle in the trap. Using an optical power of approximately 10 mW, the trap is capable of rotating micron size particles with angular velocities up to 200 radians per second and generating several hundred pN·nm of torque. The resolutions of torque measurement and angular confinement are only limited by rotational Brownian motion of the particle.

### EXAMPLES

The Examples set forth below are for illustrative purposes only and are not intended to limit, in any way, the scope of the present invention.

### Example 1

### Fabrication of Cylindrical Crystalline Quartz Optical Trapping Particles

Nanofabricated crystalline quartz cylinders that are ideally suited for torque application and detection in an angular optical trap were designed and created. The nanofabrication protocol is outlined in FIG. 6A. In particular, a 100 mm, x-cut, single crystal quartz wafer (University Wafer, Inc., South Boston, Mass.) was cleaned with hot piranha treatment. Brewer Xhric-16 antireflection coating was spun onto the reverse side of the wafer at 2500 rpm for 30 seconds, 500 R/s ramp rate, baking at 180° C. for two minutes.

The top surface was derivatized by reaction with 3-aminopropyltriethoxysilane (APTES) (Kleinfeld et al., J. Neurosci. 8:4098-4120 (1988), which is hereby incorporated by reference in its entirety). In particular, the wafer was added to approximately 15 mL of a 1% solution of APTES in 95% ethanol (95% ethanol, 5% water, pH to 5.0 using acetic acid). The wafer was sonicated in the solution for four minutes. The wafer was then removed and immersed in a 50-mL dish of 100% ethanol. This rinse was repeated two times with ethanol, sonicating the container briefly each time. The wafer was then baked at 115° C. for 20 minutes.

650 nm of OIR-620-71 photoresist was spun onto the top surface of the wafer at 1850 rpm for 30 seconds, 500 R/s. The wafer was prebaked for 120 seconds at 90° C.

10× projection UV lithography with an i-line, 365-nm stepper GCA stepper tool was used to pattern approximately 0.5 μm diameter posts into the photoresist, with a 115° C. post exposure bake for 120 seconds. Development of the wafer was achieved with 300MIF. The patterned wafer was dry etched with a trifluoromethane (50 sccm) and oxygen (2 sccm) (CHF₃/O₂) plasma for 60 minutes. Care was taken so that the photoresist was not completely etched away. Otherwise the underlying amine groups will be damaged. Residual photoresist was removed by 20 minute sonication in acetone to reveal the amino-functionalized top surface. At this point, the wafer contained approximately one billion functionalized quartz posts of nearly uniform height (1.1±0.1 μm), diameter (0.53±0.05 μm), and vertical sidewall angle (87±2 degrees) (FIGS. 6B and 6C). The homogeneity in size was limited by nanofabrication processing, primarily by the focusing and leveling capabilities of the instrument used to pattern photoresist. Some of these procedures of nanofabricating posts are similar to those previously described (Volkmuth et al., Nature 358:600-602 (1992), which is hereby incorporated by reference in its entirety).

Mechanical pressure from a microtome blade was used to remove the cylindrical quartz posts from the wafer substrate. In particular the surface was gently scraped with a clean microtome blade and the powder product was collected. The quartz posts fractured evenly at their bases (FIGS. 6D and 6E). A commercially available kit (Polysciences Inc., Warrington, Pa.) was used to covalently couple the cylinder's amino-functionalized surface to streptavidin. Note also the nanofabrication protocol outlined here can readily be modified to produce somewhat larger cylinders (e.g., cylinders of 1 μm diameter and 2 μm height) if greater torques and forces are desired.

### Example 2

### Use of Cylindrical Quartz Optical Trapping Particles in an Optical Trap

Trapping properties of the quartz cylinders were investigated using an angular optical trap.

### Angular Optical Trapping Instrument

The trapping laser (Spectra-Physics T-40, 1064 nm) was linearly polarized before it entered a 100×, 1.3 NA objective (Nikon USA, Melville, N.Y.) mounted on an inverted Eclipse TE200 microscope. The polarization angle of the input laser beam was controlled by two acousto-optic modulators with about a 100 kHz refresh rate (La Porta et al., Phys. Rev. Lett. 92:190801 (4 pages) (2004), which is hereby incorporated by reference in its entirety). The torque and angular displacement of the trapped particle were determined by a change in the angular momentum of the transmitted light as detected by a difference between in light intensities of right and left circular polarizations (La Porta et al., Phys. Rev. Lett. 92:190801 (4 pages) (2004), which is hereby incorporated by reference in its entirety). The force and linear displacement of the trapped particle were detected via a quadrant photodiode (Deufel et al., Biophys. J, 90:657-667 (2006), which is hereby incorporated by reference in its entirety).

### Angular and Linear Optical Trapping Calibrations

Torque and angle calibration methods were based on those previously described (La Porta et al., Phys. Rev. Lett. 92:190801 (4 pages) (2004), which is hereby incorporated by reference in its entirety) and are briefly summarized below. For an angularly trapped particle, the torque signal V_{τ} (in volts) was related to the particle's angular displacement 9 from the angular trap's input polarization by V_{τ}=V₀ sin(2θ). The value of V₀ was determined by rotating the input laser polarization much faster than the particle could follow, so that the input laser polarization vector scanned a quasi-stationary particle. For small angles, θ≈V_{τ}/2V₀ so that θ could be directly determined by the torque signal. Once the angle calibration was determined, angular trap stiffness k_{θ} was obtained from the angular Brownian fluctuations σ_{θ} based on the equipartition theorem:

$$\frac{1}{2} {k}_{θ} {σ}_{θ}^{2} = \frac{1}{2} {k}_{B} T ,$$

where k_{B}T is the thermal energy.

Force and linear displacement calibration methods were also based on those previously described (Deufel et al., Biophys. 1, 90:657-667 (2006), which is hereby incorporated by reference in its entirety) and are briefly summarized below. Linear displacement calibration along the direction of the light propagation (same as the cylinder axis) was measured by moving a quartz cylinder stuck on its end to the surface of the microscope coverglass through the trapping beam along this direction using a piezo stage. Once the linear displacement calibration was determined, the linear trap stiffness along the same direction was obtained from the linear displacement Brownian fluctuations σ_{z} based on the equipartition theorem:

$$\frac{1}{2} {k}_{z} {σ}_{z}^{2} = \frac{1}{2} {k}_{B} T .$$

Due to the effects of the index of refraction mismatch when using an oil immersion objective, a measured focal shift ratio of 0.76 was taken into account when determining the DNA extension (Deufel et al., Biophys. 1, 90:657-667 (2006), which is hereby incorporated by reference in its entirety).

Using the calibration procedures outlined above, repeated measurements on the same single cylinder resulted in standard deviations of 12% for both angular and linear trap stiffness calibrations. The means from different cylinders had standard deviations of 14% for the angular trap stiffness and 7% for the linear trap stiffness, resulting from slight variations in the size and shape of the cylinders.

The angular stiffness of a trapped cylinder was 11.4±1.6 nN-nm/rad (mean±standard deviation) for each Watt of laser power entering the objective; nearly 3000 pN·nm of torque could be exerted on a quartz cylinder with 0.5 W of laser power. The axial linear stiffness of a trapped cylinder was 0.59±0.04 pN/nm for each Watt of laser power entering the objective. Over 100 pN of force could be exerted on a quartz cylinder with 0.5 W of laser power. These torques and forces are well suited for studies of biological molecules.

### Example 3

### DNA Supercoiling Assay Using Cylindrical Quartz Optical Trapping Particles

Nanofabricated crystalline quartz cylinders as described in Example 1 were tested in a DNA supercoiling assay (FIG. 3). When placed in an optical trap, the cylinder naturally oriented with its functionalized and slightly smaller end towards the coverglass and therefore assumed the proper orientation for DNA attachment. In particular, the cylinder axis was made perpendicular to the optic axis of the quartz crystal and only one end of each cylinder was chemically functionalized for attachment to a DNA molecule. The cylinders were used to demonstrate direct measurement of the torque on a DNA molecule as it underwent a phase transition from B-form to supercoiled P-form during DNA supercoiling.

When a DNA molecule is positively supercoiled under moderate constant tension (approximately 4-28 pN), the DNA is expected to undergo a phase transition from B-form to supercoiled P-DNA (scP-DNA) (Bryant et al., Nature 424:338-341 (2003); Strick et al., Biophys. J. 74:2016-2028 (1998), which are hereby incorporated by reference in their entirety). The onset of the phase transition should be marked by an abrupt plateauing of torque. In these experiments, a linear 2.1 kbp dsDNA segment was ligated to a 62-bp, 6-biotin-tagged oligomer at one end, and a 62-bp, 6-digoxygenin-tagged oligomer at the other end. The multiple tags at each end ensured that the ends of the DNA were torsionally constrained at both the streptavidin coated end of the quartz cylinder and the anti-digoxygenin coated coverglass. The dsDNA molecule was tethered in PBS and then held under 10 pN of tension. Positive twist was then added to the dsDNA molecule at a rate of 2 turns/second, while a computer-controlled servo loop feeding back on a piezoelectric stage maintained constant tension in the dsDNA molecule. Five signals were simultaneously recorded: axial force, axial displacement of the cylinder from the trap center, the axial position of the piezo, torque, and the angular displacement of the optic axis of the cylinder from the angular trap center. Data were anti-alias filtered at 1 kHz, digitized at 2 kHz, and averaged with a 1.5 second moving window to reduce Brownian noise.

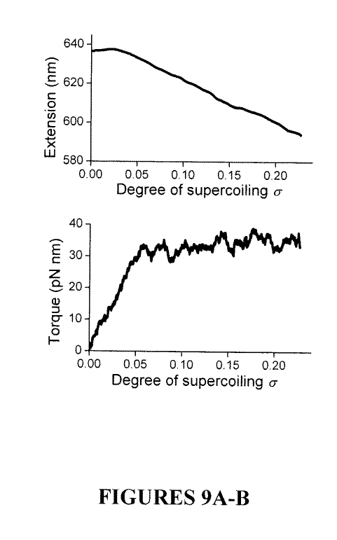

Both the torque and DNA extension were measured as functions of the degree of supercoiling σ, defined as the number of turns added to dsDNA divided by the number of naturally occurring helical turns in the given dsDNA (FIGS. 9A-B). At low σ values (0.00-0.05), the DNA exhibited a nearly linear increase in torque with σ. Over this range of σ, the DNA is expected to adopt the canonical B-DNA form. Once σ reached 0.05, the torque began to plateau at approximately 33 pN nm, indicating the beginning of a phase transition. These critical σ and critical torque values are consistent with previous measurements, and have been interpreted as indicative of the B-DNA to scP-DNA transition (Bryant et al., Nature 424:338-341 (2003), which is hereby incorporated by reference in its entirety). A slight increase in extension was also observed for σ values in the range 0.00-0.03. This has been attributed to a negative twist-stretch coupling (Lionnet et al., Phys. Rev. Lett. 96:178102 (4 pages) (2006); Gore et al., Nature 442:835-839 (2006), which are hereby incorporated by reference in their entirety).

These results demonstrate that nanofabricated quartz cylinders are well suited for precision measurements in an angular optical trap. For the first time, torque, angle, force, and DNA extension can be simultaneously monitored at kHz rates. This capability will allow for future detection of rapid events and concurrent observation of the linear and angular behaviors of DNA. The cylinders should provide a powerful tool for the investigation of torsional properties of biopolymers and rotational motions of biological molecular motors.

### Example 4

### Testing of Buckling Transition During Plectoneme Formation in Individual DNA Molecules

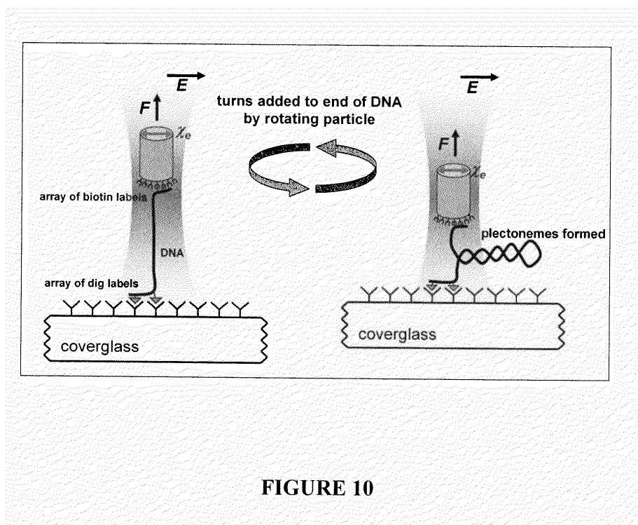

Here experiments were carried out to measure the response of DNA as it was overwound to introduce positive supercoils. The experimental procedure resembles that previously used for magnetic tweezers studies (Strick, Science, 271:1835-1837 (1996), which is hereby incorporated by reference in its entirety), but with the addition of direct torque measurement. During an experiment as shown in FIG. 10, one end of a DNA molecule was torsionally constrained to the end of a cylinder and the other end to the surface of a microscope coverslip. The cylinder was first moved away from the coverglass to stretch the DNA molecule along the axial direction (i.e., the direction of laser propagation) (Deufel et al., Biophys. 1, 90:657-667 (2006), which is hereby incorporated by reference in its entirety). When the force reached a preset value, it was then held constant via modulation of the position of the coverglass. Subsequently DNA was overwound by steady rotation of the cylinder via rotation of the input laser polarization under a constant force. During this time, torque, angular orientation, position, and force of the cylinder as well as the location of the coverglass were simultaneously recorded. All experiments were performed in PBS with 150 mM NaCl at 23.5° C.

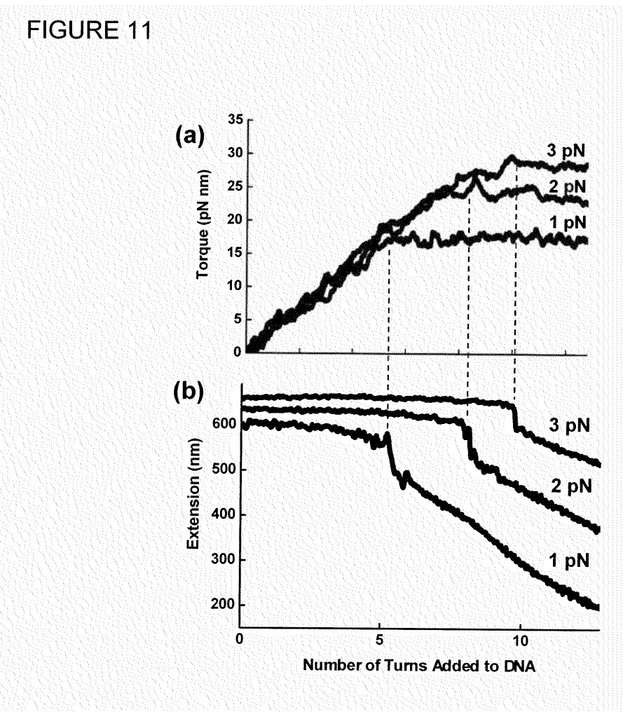

FIGS. 11A-B depict representative single traces of torque and extension as functions of number of turns added to the DNA at three different applied forces (1, 2, and 3 pN). The experiment began with a torsion-free DNA molecule. As DNA was overwound at 1 turn/second, torque increased linearly while the extension remained approximately constant. This continued until the DNA buckled to form a plectoneme, indicated by a sudden decrease in extension. The buckling transition arises when the free energy of the extended DNA becomes larger than that of the initial plectonemic structure within the DNA. Beyond this transition, plectonemes were formed continuously with additional twist. With each additional turn in this region, a single plectoneme is expected to form along the molecule (Strick, Science, 271:1835-1837 (1996), which is hereby incorporated by reference in its entirety). As the DNA molecule was converted from extended to plectonemic domain, torque maintained a constant value over the observed region, while the extension decreased linearly. Note that the torque and the extension slope after buckling were all strongly sensitive to applied tension in the DNA. Additional experiments were carried out to verify that the data were taken under quasi-equilibrium conditions; when the supercoiled DNA was relaxed at the same rotation rate as was used for the generation of supercoiled DNA, data were essentially indistinguishable.

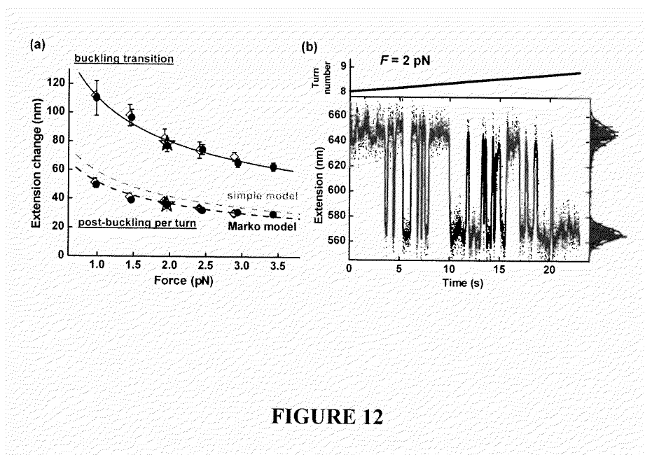

One of the most significant features of the overwinding data in FIG. 11B is the pronounced sharp drop in extension at the buckling transition, corresponding to the formation of the initial plectoneme. Interestingly such an abrupt transition was absent in previous magnetic tweezer measurements where instead a smooth and gradual transition was observed (Strick, Science, 271:1835-1837 (1996), which is hereby incorporated by reference in its entirety). The angular trapping method allowed detection of the abrupt transition, likely due to higher bandwidth and increased spatial resolution together with the use of shorter DNA tethers. As shown in FIG. 12A, the magnitude of the extension drop observed at the buckling transition was dependent on the applied force, following a power law of ˜F^{-0.5}. In contrast, the extension decrease per turn after the transition followed a power law of ˜F^{-0.4}, as observed in previous magnetic tweezer studies (Strick et al., Rep. Prog. Phys., 66:1-45 (2003), which is hereby incorporated by reference in its entirety). Furthermore, the first plectoneme was approximately twice as large as the subsequent plectonemes. This indicates that the initial plectoneme was able to absorb more extension than a subsequent plectoneme in the helical coil. These two distinct regimes of extension change versus force are clearly depicted in FIG. 12A. In addition, three different DNA templates were used for these experiments: a 2.2 kbp DNA, a 4.2 kbp DNA containing the 2.2 kbp sequence, and a 2.2 kbp DNA with a sequence entirely different from the first two. The measured extension changes were found to be the same for all three DNA templates, indicating that they are neither length nor sequence dependent within the resolution limits of our instrument.

Data in FIG. 11 suggest that the energy barrier between the extended and plectonemic states is low enough for transitions between the two states to occur at an observable rate. To test this idea, a DNA tether was held at 2 pN, and overwound extremely slowly (0.04 turn/second) through the buckling transition (FIG. 12B). The extension of the DNA was observed to fluctuate between two discrete states, corresponding to pre- and post-buckling. The rates of fluctuation were highly sensitive to twist and it is estimated that they were on the order of approximately 10 Hz near the rotational mid-point of the transition. The two states were separated by 79 nm, in good agreement with the extension drop observed at the same force in the rapid winding experiment in FIG. 12A.

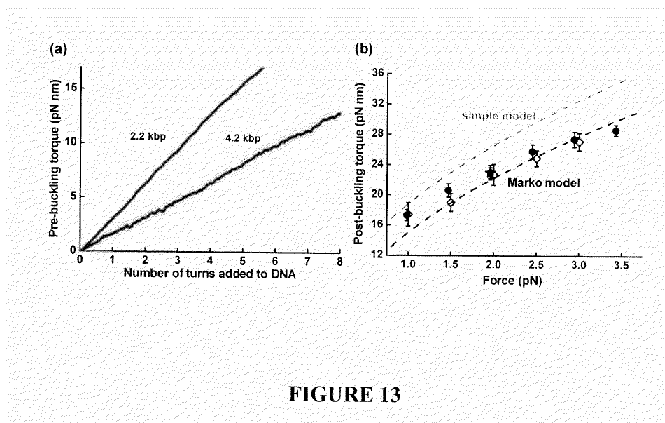

Measurements like those shown in FIG. 11A allowed direct determination of the torsional modulus of DNA. Torque-turn relations were plotted by pooling the torque data taken at various forces prior to buckling for either the 2.2 kbp or 4.2 kbp tethers (FIG. 13A). The torque-turn relations showed linear relations and scaled with the length of the DNA. Prior to buckling, DNA may be modeled as a simple elastic torsional rod. As twist is applied to the DNA, the restoring torque τ will increase linearly with the twist angle, as given by:

$$τ = C \frac{2 π n}{{L}_{0}} ,$$

where L₀ is the contour length of the rod with 1 bp corresponding to 3.38 nm, n is the number of turns added, and C is the torsional modulus. The slopes of the measured torque-turn relations yielded a torsional modulus of C=90±3 nm k_{B}T (88±4 nm k_{B}T) for the 2.2 kbp (4.2 kbp) DNA. Previous studies, which have employed techniques such as DNA cyclization (Horowitz et al., J. Mol. Biol., 173:75-91 (1984), which is hereby incorporated by reference in its entirety), fluorescence polarization anisotropy (Selvin et al., Science, 255:82-85 (1992), which is hereby incorporated by reference in its entirety), or magnetic tweezers (Strick et al., Genetica, 106: 57-62 (1999), which is hereby incorporated by reference in its entirety), have reported values ranging from 50 to 120 nm k_{B}T. These measurements fall well within this range, and represent the first time torque has been directly measured on single DNA molecules held at physiologically attainable tensions. The measured twist modulus corresponds to a twist persistence length

$$\frac{C}{{k}_{B} T}$$

of approximately 90 nm

Measurements like those depicted in FIG. 11A also allowed direct determination of the post-buckling torque. FIG. 13B summarizes data for post-buckling torque versus force for all three DNA templates. The post-buckling torque increased with force and this relation was independent of DNA length and sequence.

A number of models exist to explain plectoneme formation in DNA post-buckling. A simple model treats DNA as an elastic rod and assumes that each plectoneme formed is circular (Strick et al., Rep. Prog. Phys., 66:1-45 (2003), which is hereby incorporated by reference in its entirety). This simple classical rod model predicts that the extension change per turn after buckling is

$$Δ z = π \sqrt{\frac{2 {L}_{p} {k}_{B} T}{F}}$$

and the post-buckling torque is τ_{c}=\sqrt{2Lₚk_{B}TF}, where Lₚ is the persistence length of the DNA, k_{B}T is the thermal energy, and F is the applied force. Force-extension measurements similar to those described before (Wang et al., Biophys. 1, 72:1335-1346 (1997), which is hereby incorporated by reference in its entirety) were carried out and it was determined that Lₚ=43±3 nm under these experimental conditions. The predicted post-buckling extension change per turn and post-buckling torque versus force, shown FIGS. 12A and 13B respectively are greater than measurements by as much as 25%.

Several more elaborate models exist to describe DNA supercoiling analytically (Marko, Phys. Rev. E, 76:021926 (13 pages) (2007); Bouchiat et al., Phys. Rev. Lett., 80:1556-1559 (1998); Purohit et al., Phys. Rev. E., 75:039903 (1 page) (2007), which are hereby incorporated by reference in their entirety). In particular, an elegant recent theoretical work by John Marko (Marko, Phys. Rev. E, 76:021926 (13 pages) (2007), which is hereby incorporated by reference in its entirety) employed a detailed statistical mechanics analysis to incorporate an effective torsional flexibility of the plectonemic state (Vologodskii et al., J. Mol. Biol., 227:1224-1243 (1992), which is hereby incorporated by reference in its entirety) and a force-dependent torsional flexibility of the extended state (Moroz et al., PNAS, 94:14418-14422 (1997), which is hereby incorporated by reference in its entirety). This model, which is referred to here as the Marko model, provides closed-form expressions for both the extension change per turn and the post-buckling torque. All parameters in the model were experimentally determined in this work, except for the plectonemic rigidity. A global fit of our measurements to the model was performed using the plectonemic rigidity as the single fit parameter. The resulting best fit for the extension change per turn was in excellent agreement with the measurements (FIG. 12A) and the resulting best fit for the post-buckling torque agreed with the measurements to within 15% (FIG. 13B). This good agreement lends strong support to the Marko model. In addition, the best fit value for the plectonemic rigidity was 26 nm, within the range of 21-27 nm as previously estimated (Vologodskii et al., J. Mol. Biol., 227:1224-1243 (1992), which is hereby incorporated by reference in its entirety).

We are not aware of any analytical models suitable for prediction of the observed extension change and dynamics at the buckling transition. In principle these can be achieved using Monte Carlo calculations (Vologodskii et al., Biophys. J., 70:2548-2556 (1996), which is hereby incorporated by reference in its entirety). Mechanical rod models should also be extendable to explain DNA supercoiling. Goyal et al. formulated a non-linear dynamic rod model which shows an abrupt buckling followed by subsequent formation of plectonemes in macroscopic rods (Goyal et al., J. Comp. Phys., 209:371-389 (2005), which is hereby incorporated by reference in its entirety), a prediction that bears much resemblance to our measurements.

The highly dynamic nature of a twisted DNA molecule at the buckling transition may have important biological consequences in vivo. The specific supercoiling density (0.00-0.10) and applied force (1.0-3.5 pN) are well within the range commonly experienced by DNA in the cell. If a DNA molecule is subject to moderate stresses, distant elements on the sequence may transiently be brought into contact, which may facilitate the binding of DNA looping proteins or transcription factors (Nelson, Proc. Nat. Acad. Sci., 96:14342-14347 (1999), which is hereby incorporated by reference in its entirety). The rapid formation and loss of these transient loops would therefore greatly reduce the search time needed for a protein to find two spatially separated sequence elements on the template.

Direct measurements of DNA torsional response lays an important foundation for the understanding of many biological processes that are regulated by torque. For example, topoisomerases are known to mediate linking numbers in DNA by sensing torsional stress in the DNA (Koster et al., Nature (London) 434:671-674 (2005); Strick et al., Nature 404:901-904 (2000), which are hereby incorporated by reference in their entirety). RNA polymerases as well as other groove-tracking enzymes are expected to rotate about the DNA helical axis (Harada et al., Nature (London) 409:113-115 (2001); Revyakin et al., Science, 314:1139-1143 (2006), which are hereby incorporated by reference in their entirety), and would thereby generate and move against positive torque in the downstream DNA. The presence of torque is also expected to regulate nucleosome stability which in turn regulates gene expression. We anticipate major progress in these areas with the advent of a number of biophysical techniques including the one presented here to rotate microscopic particles and measure their rotational motions (Deufel et al., Nat. Meth., 4:223-225 (2007); Bryant et al., Nature (London), 424:338-341 (2003); Bishop et al., Phys. Rev. Lett., 92:198104 (4 pages) (2004); Oroszi et al., Phys. Rev. Lett., 97:058301 (4 pages) (2006), which are hereby incorporated by reference in their entirety). The angular optical trap, with its wide bandwidth, high spatial resolution, and ability to simultaneously measure force and torque should prove to be a valuable tool to understand these highly kinetic and mechanical processes.

### Example 5

### Optimization of Optical Trapping Particles

Optical trapping is a powerful technique used to investigate the mechanical properties of the molecular motors that govern cellular processes. In order to examine such mechanisms, trappable “handles” must be developed that can be used for attachment to biological samples. This example involves the design and fabrication of cylindrical trapping particles to be used in measuring forces and torques exerted on DNA, in addition to optimizing existing fabrication protocols. In previous examples, the entire end of a cylinder was chemically functionalized for binding to DNA. In this example, the functionalized area was dramatically reduced to prevent the unwanted precessing of a cylinder in an optical trap, thereby minimizing measurement noise.

In this example, crystalline quartz was used, which is birefringent, as the substrate for fabricating cylindrical trapping particles in order to make measurements of torque and force on DNA in an angular optical trap.

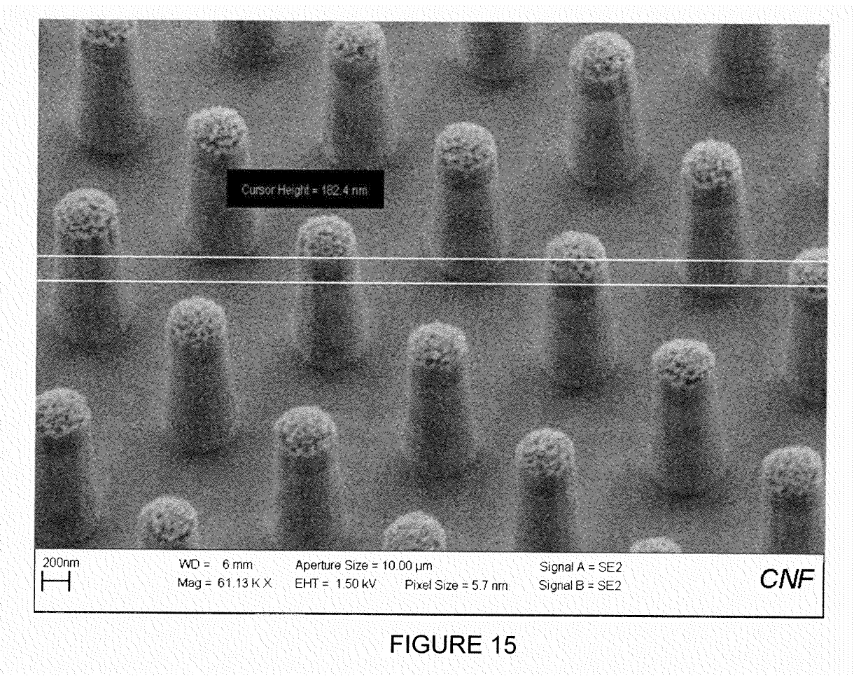

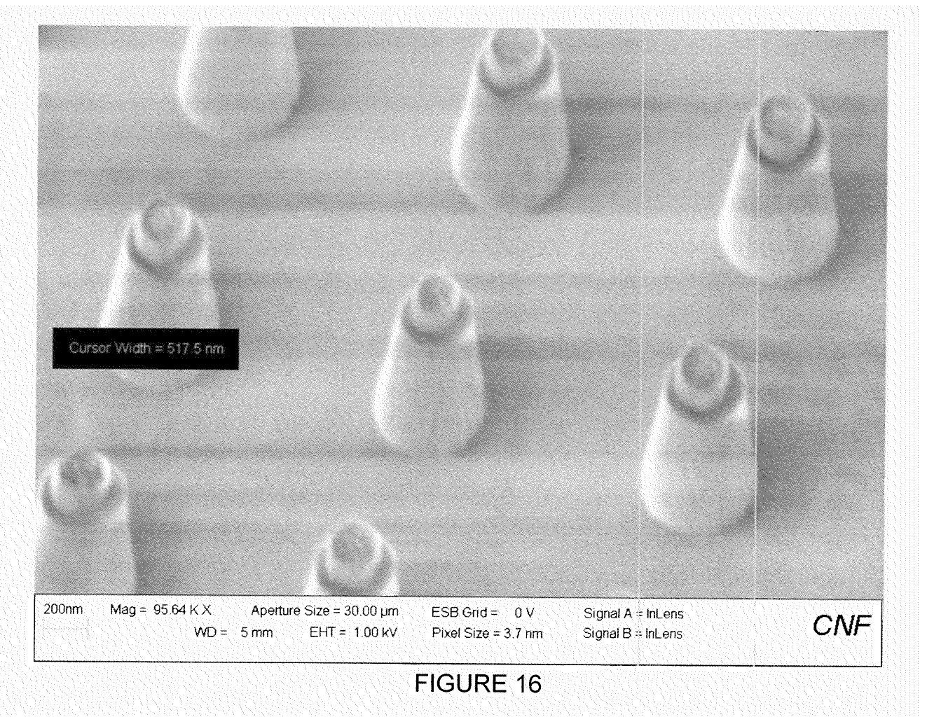

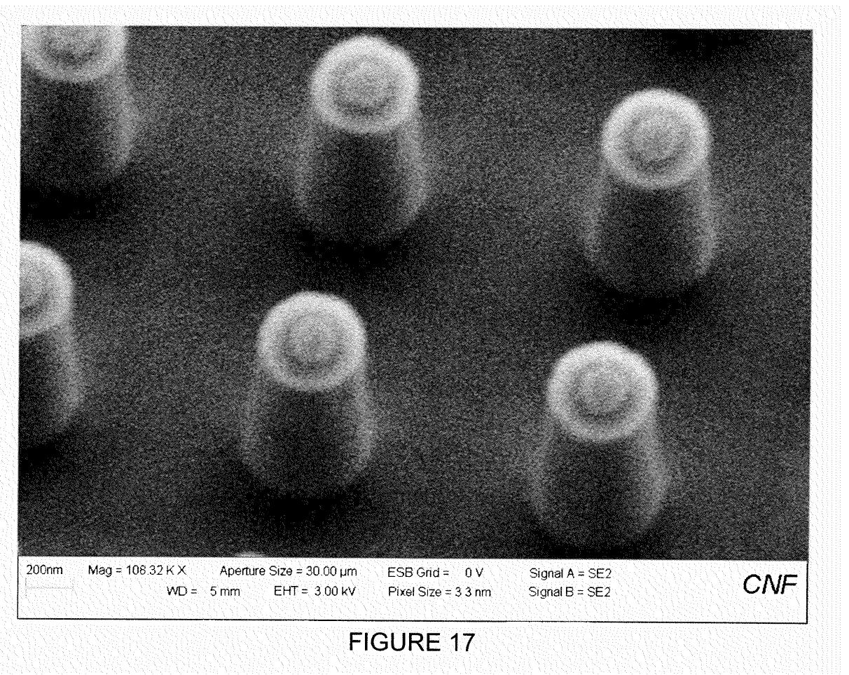

The primary purpose of this example was to optimize the existing cylindrical nanoparticle fabrication protocols. FIG. 14 outlines the optimized fabrication protocol. Step A pictures the initial step in this protocol; here a thin anti-reflective coating (ARC) has been applied (this coating was simply there to prevent unwanted ring like structures from appearing on the wafer from the reflective chucks used in the exposure process). In Step B the top surface was reacted with 3-aminopropyltriethoxysilane (APTES); this is the functionalized area to be reduced. Approximately 660 nm of OIR 620-71 was spun onto the wafer in Step C. A 10× stepper was used to expose the pattern in Step D. Step E depicts the first anisotropic dry etch (CHF₃/O₂ used as etching gas) (see FIG. 15). Step F ultimately leads to a reduction in the functionalized area, which is the aim of this entire protocol; here an isotropic dry etch is performed in which the resist is, for the most, symmetrically etched away (see FIG. 16). A second anisotropic etch is performed in Step G; this creates a 10 nm step in the quartz and provides assurance that all of the unprotected APTES is removed. In the next step, Step H, the resist was removed by sonicating the cylinders/wafer in an acetone solution (see also FIG. 17). The cylinders, in Step I, have simply been cleaved using a microtome blade. Finally, Step J depicts the end product; the localized area of APTES on top of the cylinders just has to react with streptavidin for attachment to a DNA molecule in an optical trap.

With the optimized protocol, the functionalized area was reduced by approximately 80% when compared to the original protocol (the functionalized area went from about π×(450 nm)² to about π×(200 nm)²). This significant decrease in area has been shown significantly reduce unwanted abnormal precessing of the cylinder in the optical trap, and thus gave more accurate measurements.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

We claim:

1-24. (canceled)

25. An angular optical trap system comprising:
a sample chamber;
an optical trapping particle comprising: (1) a birefringent crystalline particle having a body and a length extending between a first end and a second end, said particle comprising an optic axis perpendicular to the length of the body;
an angular optical trap assembly comprising a laser, a laser polarization rotator, and an input polarization detector, wherein the laser is positioned to generate an input trapping beam that passes through the laser polarization rotator to generate a first output trapping beam, wherein a first portion of the first output trapping beam passes into the input polarization detector and a second portion of the first output trapping beam passes into the sample chamber.

26. The angular optical trap system according to claim 25, further comprising:
a detection device positioned to receive a second output trapping beam from the sample chamber.

27. The angular optical trap system according to claim 26, wherein the detection device is a force/position detector, a torque/angle detector, or both.

28. The angular optical trap system according to claim 25, wherein the length of the body is greater than the largest width dimension of the first or second ends.

29. The angular optical trap system according to claim 25, further comprising:
a target molecule or attachment device attached at a first position to the first or second end of the optical trapping particle to form a complex, wherein the complex is positioned within the sample chamber.

30. The angular optical trap system according to claim 29, wherein the optical trapping particle complex comprises the target molecule and a substrate, wherein the target molecule is attached at a second position to the substrate.

31. The angular optical trap system according to claim 30, wherein the target molecule comprises a T-shaped portion suitable for attaching to the optical trapping particle, substrate, or both.

32. The angular optical trap system according to claim 29, wherein the target molecule is a nucleic acid molecule, a protein molecule, a polypeptide, or an organic polymer.

33. The angular optical trap system according to claim 32, wherein the nucleic acid molecule comprises ribonucleotides, deoxyribonucleotides, modified ribonucleotides, modified deoxyribonucleotides, peptide nucleic acids, modified peptide nucleotide analogues, modified phosphate-sugar backbone oligonucleotides, nucleotide analogues, or mixtures thereof.

34. The angular optical trap system according to claim 29, wherein the attachment device is a propeller, drill, polisher, grinder, mill, or gear.

35. The angular optical trap system according to claim 25, wherein the optical trapping particle comprises a material selected from the group consisting of quartz, sapphire, mica, calcite, corundum, beryl, rutile, tourmaline, calomel, lithium niobate, magnesium fluoride, ruby, peridot, zircon, topaz, olivine, perovskite, and nepheline.

36. (canceled)

37. The angular optical trap system according to claim 25, wherein the optical trapping particle has a cross-sectional shape that is circular, elliptical, or polygonal.

38. The angular optical trap system according to claim 37, wherein the polygonal cross-sectional shape is selected from the group consisting of a triangle, a square, a trapezoid, a rectangle, a parallelogram, a pentagon, a hexagon, a star shape, and a polygon having seven or more sides.

39. The angular optical trap system according to claim 25, further comprising:
a functional group on the first or second ends capable of coupling to a target molecule or attachment device.

40. The angular optical trap system according to claim 39, wherein the functional group is an olefin, amino, thiol, hydroxyl, silanol, aldehyde, keto, halo, acyl halide, or carboxyl group.

41. The angular optical trap system according to claim 25, wherein a center portion of the first or second end comprises the functional group capable of coupling to the target molecule or attachment device.

42. The angular optical trap system according to claim 25, wherein the body is tapered from the first end to the second end such that the surface area of the first end is larger than the surface area of the second end.

43. The angular optical trap system according to claim 25, wherein one or more optical trapping particles and the angular optical trap assembly are configured to generate multiple angular optical traps.

44-60. (canceled)
